(19) United States
(12) Patent Application Publication
(10) Pub. No.: US 2025/0099340 A1
(43) Pub. Date: Mar. 27, 2025

(54) Adult Enteral Feeding System

(71) Applicant: Robert L. Palmerton, Edmond, OK (US)

(72) Inventor: Robert L. Palmerton, Edmond, OK (US)

(21) Appl. No.: 18/474,739

(22) Filed: Sep. 26, 2023

(51) Int. Cl. A61J 15/00; A61M 5/142; A61M 5/168

(52) U.S. Cl. CPC A61J 15/0076; A61J 15/0088; A61M 5/14212; A61M 5/16877; A61M 2205/3365; A61M 2205/3389

(57) ABSTRACT

A high rate pump system for tube feeding having at least a liquid reservoir, a motorized pump with a power supply and at least an on/off control, a tube-feeding connector; and at least one segment of tubing in liquid communication from the reservoir through the pump to the connector, wherein the pump, power supply, and at least one tubing segment are configured to deliver over one liter of liquid nutritional product per hour from the reservoir to the connector.

### FIELD OF THE INVENTION

The present invention relates to systems to pump and deliver liquid sustenance to adult patients.

### BACKGROUND OF THE INVENTION

As the result of a variety of medical conditions, injuries, and diseases, many adult patients receive nutrition through a liquid feeding apparatus generally known as gastrostomy feeding tubes (“G-tube” or “button”) and devices (delivered directly to the stomach from an outside port or external flange), including percutaneous endoscopic gastrostomy (PEG), laparoscopic or open gastrostomy or radiologically inserted gastrostomy (RIG). Still other patients may receive their sustenance via a nasogastric feeding tube (“NG-tube”) which leads from the nostrils down the throat and into the stomach, a nasojejunal feeding tube (“NJ-tube”) which leads from the jejunum up and into the stomach, gastrojejunal feeding tube (“GJ tube”) which delivers liquid nutrition to both the stomach and jejunum, and jejunal feeding tubes (“J-tubes”). One can generalize that, for various reasons related to the patient, all of these feed tube types bypass one or more biological elements of eating drinking to deliver readily metabolized liquid nutrition products to one or more stages within the digestion tract by means of a tube, port, flange.

While some patients may receive their liquid nutrition slowly and passively through a gravity-driven system, such as an elevated bag with a tube between the bag and the port or flange, other patients receive it through a pump device or a syringe style device.

### SUMMARY OF THE INVENTION

A high rate pump system for tube feeding is disclosed having at least a liquid reservoir, a motorized pump with a power supply and at least an on/off control, a tube-feeding connector; and at least one segment of tubing in liquid communication from the reservoir through the pump to the connector, wherein the pump, power supply, and at least one tubing segment are configured to deliver over one liter of liquid nutritional product per hour from the reservoir to the connector. One or more optional features are disclosed.

### BRIEF DESCRIPTION OF DRAWINGS

The figures presented herein, when considered in light of this description, form a complete disclosure of one or more embodiments of the invention, wherein like reference numbers in the figures represent similar or same elements or steps.

FIG. 1 provides a schematic of an example embodiment according to the present invention of a high-delivery-rate feed pump.

FIG. 2 illustrates another example embodiment according to the present invention of a high-delivery-rate feed pump having a removable reservoir.

FIG. 3 provides more details of an example embodiment according to the present invention of a removable reservoir for a high-delivery-rate feed pump.

FIG. 4 sets forth an example embodiment according to the present invention of control logic, such as circuitry or micro-controller-based logic, for a high-delivery-rate feed pump

### DETAILED DESCRIPTION

The present inventor(s) have recognized and solved certain unmet needs in the arts relating to delivery and consumption of liquid nutrition products via the variety of tube-feeding devices commonly used by patients today. Most liquid nutrition is received via commercially-manufactured liquids which are supplied via bottles for home and non-institutional use, while some liquid nutrition is delivered by bags similar to “IV bags” in clinical and hospital use or for home use with patients who are immobilized (e.g., confined to beds or wheel chairs, etc.).

While the delivery of thin liquids such as water, saline solution and even glucose solution may be performed in this manner, especially to hydrate the patient and/or to rinse or flush the tubes (lines) after a feeding session, most of the commercially-available liquid nutrition products are much thicker than aqueous solutions, for which pumps, valves, clamps, tubes, flanges, buttons, fittings, etc., must be specifically designed to accommodate the thickness, consistency and weight.

Available pumps for tube feeding tend to be very similar in performance, being mechanically designed to sit on a table top (for home or office use), mounted on a pole (for bedside use), or sometimes portable with rechargeable batteries in a backpack (for use while traveling or away from home). And, most available pumps are designed for use by all of the following: infants, juveniles, teens, adults, immobilized users and fully mobile users. Because the smallest of users have the smallest of stomachs and jejunums, and because immobilized users may not receive and process thick liquid nutrition as quickly as fully mobilized users may, the delivery rate of available pumps tends to not exceed 999 ml/hr for a bolus rate, and often much slower for a continuous feed rate.

The present inventor has realized, however, that these slow rates of feeding are not only unnecessary for adult users who have full mobility and whose bodies are capable of receiving greater amounts of liquid nutrition in shorter amounts of time than children and/or immobilized users, such rate-limited designs are highly inconvenient for many users who wish to have this aspect of their lives less impactful. For example, consider a cancer patient adult who can no longer take solid food by mouth due to mouth, jaw or throat restrictions after cancer treatment, but who is otherwise fully mobile. Such an adult may carry on an otherwise normal daily life, but when meal time arises, breaking away from social engagement for an hour or more to slowly receive liquid nutrition may not only be inconvenient, it may cause loss of business opportunity (conversations had in the absence of the patient), and mental and mood effects of social isolation. For example, an fully mobile adult male may require three cartons of a liquid nutrition, per meal, such as ISOSOURCE HN [™] available from Nestle HealthScience of Bridgewater, New Jersey, USA, at 250 ml per carton, for a total of 750 ml per meal. At a maximum bolus rate of the available pumps on the market of 0.999 ml/hr, it can require 45 minutes to pump the liquid nutrition, plus additional time for preparation, clean up, dressing, undressing, etc. This extended period of time may not be easily accommodated by more active users, and as such, some users may forgo taking their nutrition as recommended during the day, reserving such intrusive activities for early in the morning and later at night. By essentially taking three meals in the times of two meals, this may cause certain nutritional deficiencies, blood glucose spikes and slumps, etc.

As such, the present inventor, being such a patient himself, set upon finding a suitable feeding pump for those who are not restricted in the volume or rate with which they may receive liquid nutrition. Having found no such available pump, the present inventor has devised the present invention, a plurality of example embodiments of which are disclosed in the following paragraphs and drawings. It will be disclosed with regarding to particular feeding tube system and the foregoing ISOSOURCE HN [™] liquid nutrition product, but those ordinarily skilled in the arts will readily recognize that other embodiments may be realized within the spirit and scope of the present invention for use with other nutritional products and for all types of feed tube arrangements, fixtures, and therapies, including but not limited to the aforementioned feed tube systems.

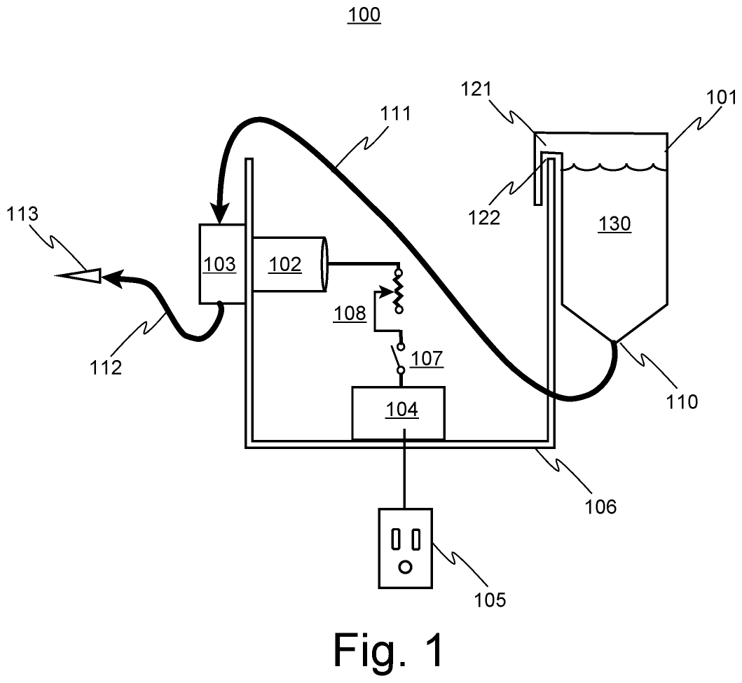

Turning now to FIG. 1, a example embodiment 100 of a feeding pump according to the present invention is shown. It has a reservoir 101 for holding liquid nutritional product, a pump 103 and motor 102 for drawing the liquid nutritional product from the reservoir and delivering at pressure to a feeding tube connector 113 via one or more segments of tubing 111, 112. It has a power supply, such as an AC to DC adapter 105, a battery 104, with one or more controls such as an on/off switch 107 and a flow rate adjustment 108, that controls the motor speed and, in turn, the delivery rate of the liquid nutritional product to the patient or user. This particular example embodiment 100 includes a frame 106 upon which the motor, pump, power supply and controls are mounted, and also upon which the reservoir 101 may removeably hang via a hook, notch, groove, or other suitable mating retainer set 121, 122.

Use of this system is simple—the user prepares his or her port or flange for use, then connects the connector 113 to their feeding port or flange. The user also fills the reservoir with the desired liquid nutritional product 130, which is conveyed out a drain 110 of the reservoir into the tubing segments 111, 112, through the pump 103, to the connector 113 when the power switch 107 is ON at the rate controlled by the optional rate control 108.

Upon feeding completion, the power switch 107 is turned off, a clamp may be applied to the tubing segments, and the connector 113 is disengaged from the user's flange or port. Optional flushing of the port may be accomplished using a syringe, per usual practices.

In this particular example embodiment, a prototype using a peristaltic pump, a model GB37-530 peristaltic pump with motor, such as the “Volume flow rate dc motor or stepper peristaltic pump” available from Shanghai RobotDigg Equip Makers of Moushan Town, Yuyao of Ningbo, China, is able to deliver the 750 ml meal in just a matter of a few minutes using a standard tubing size for feeding systems. At an inside diameter tube of 6.8 mm, such pumps are able to deliver 400 ml per minute, or 750 ml in 1.8 minutes full speed, for example. Fittings, curves, thickness of the liquid, etc., may reduce this delivery rate, but it is still within just 5-10 minutes rather than the 45 minutes using the slow delivery pumps available in the art. It should be noted that, in this example embodiment, a peristaltic pump is used because no part of the pump comes in contact with the food product, as the food product stays within the tube from reservoir to connector, and the pump merely compresses the tube in a sequence to cause flow in one direction or another. Not only does this protect the food from contamination, it also provides for easier and more complete clean-up after the feeding. The pump portion of the peristaltic pump can be disassembled and the section of tubing which is compressed can easily be replaced in most models. Peristaltic pumps are self-priming, as well.

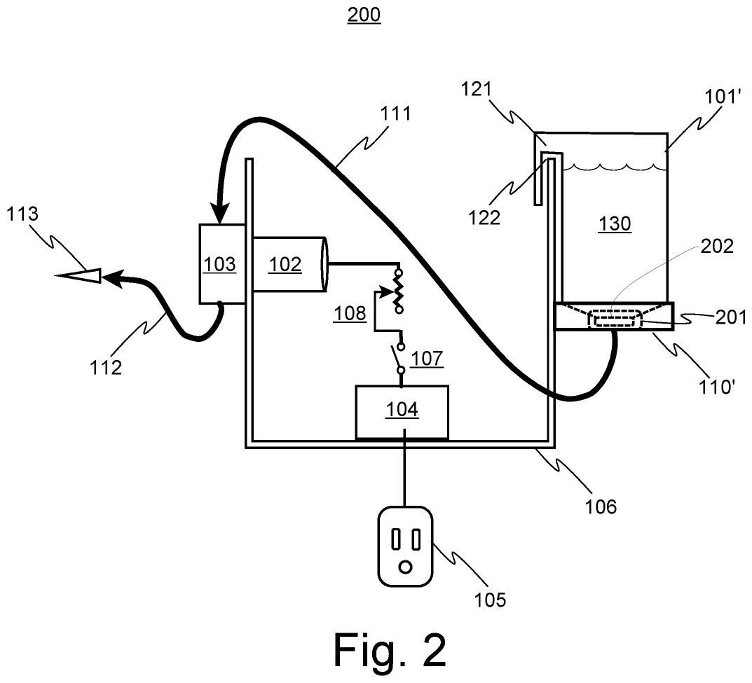

Turning now to FIG. 2, another example embodiment 200 of the present invention is shown, which is similar to that of FIG. 1, but which has a dockable reservoir 101′. Rather than having a static tubing connection at the bottom of the gravity fed reservoir, this embodiment has a valve system 201, 202 in a dock 110′ which, when the reservoir is received into the dock, the valve system is opened to allow free flow of the liquid nutritional product from the reservoir to the one or more tubing segments 111, 113. When the reservoir is removed from the dock 110′, the reservoir valve 202 automatically closes to prevent the liquid nutritional product from pouring out of the disconnected, undocked reservoir. This allows for easier loading (pouring liquid nutritional product from the carton into the reservoir), unloading (discarding unused liquid nutritional product), and optionally storage of unused liquid nutritional product. While a variety of such docking valves are contemplated as effective for various embodiment, a valve such as those typically found at the bottom of removeable water reservoirs for coffee makers and clothing steamers is a good candidate for this element of this particular embodiment. More details of such docking valves can be readily found in the art, such as in International Patent Application WO 2014/078297 A1 by Harvey Levine, et al., published by the World Intellectual Property Organization (WIPO) on May 22, 2014. Embodiments of the present invention such as this provide for convenient handling of the liquid nutritional product.

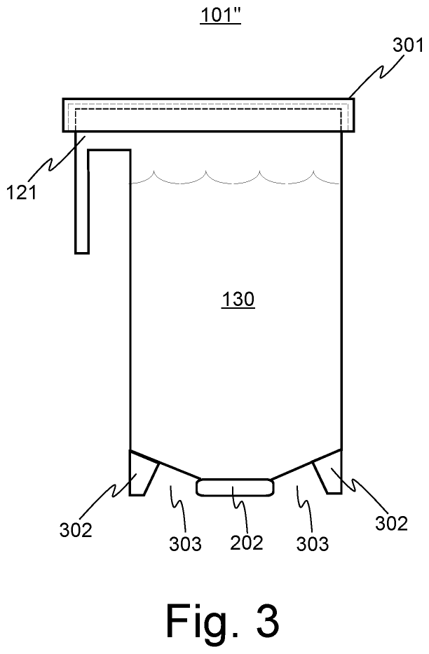

Referring now to FIG. 3, another example embodiment according to the present invention of a reservoir 101″ is shown which includes a docking valve 202 disposed at the bottom of the reservoir, as well as being provided with one or more stand 302 structures, such as feet or legs, which prevent the vertically-oriented undocked reservoir from tipping over when rested on a table top or in a refrigerator on a shelf, and a defined space 303 for receiving a dock. A lid 301, such as a snap on or screw on (threaded) lid, may be provided to prevent leakage and preserve the integrity of unused liquid nutritional product 130 until the next feeding.

Due to the self-priming nature of some pumps, such as peristaltic pumps, it may not be necessary to use a reservoir with a funnel shaped bottom and a gravity-fed drain to assist in priming the pump. In some embodiments, the end tube segment extending from the reservoir to the pump may be dropped into or dipped into the liquid nutritional product in the reservoir, and later during flushing and clean-up, the end of the tube can simply be moved to a reservoir containing clean water.

In another embodiment, the pump arrangement according to the invention, such as those shown in FIGS. 1 and 2, may be realized without a reservoir. In such embodiments, the end of the tube segment for receiving the liquid nutritional product may be essentially open or disconnected, such as covered by a cap and optionally provided with a small weight at the end. In this manner, the end of the tube may be dropped into any container, and especially into an open carton of liquid nutritional product, from which the pump may directly draw. This may also allow dropping the end of the tube into a container of clean water for flushing or hydration of the user.

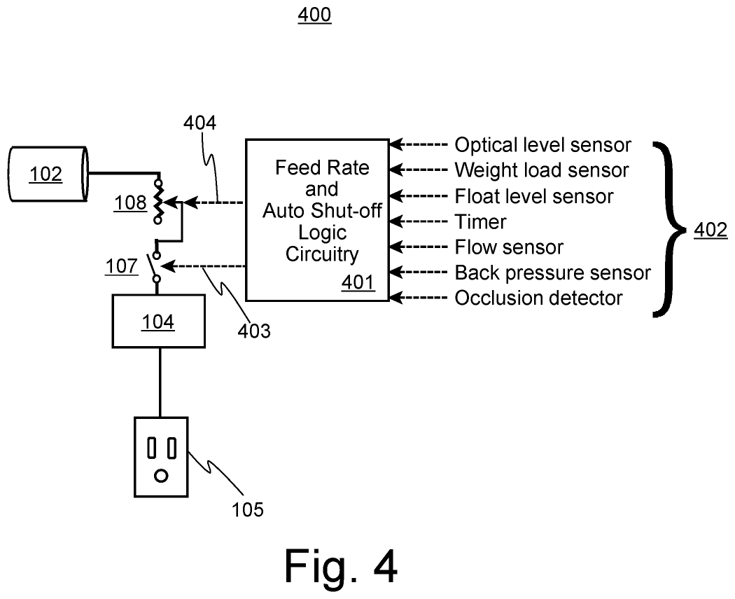

FIG. 4 shows 400 example control logic according to one or more embodiments of the present invention. In this arrangement, the feed rate and/or automatic shut-off logic 401 receives one or more inputs from various sensors 402. According to the logic 401, the on/off switch 107 may be controlled 403 and the optional flow rate control 108 may be automatically adjusted 404. Such control logic may be realized in a variety of forms, such as discrete logic circuits, embedded microcontroller circuitry with associated firmware, mechanical controls, or a combination thereof.

For example, based on a received input from a sensor for the level of the liquid nutritional product remaining in the reservoir, such as but not limited to an optical level sensor, a weight level sensor, a float level sensor or a timer, the control logic may shut off the feed pump automatically (when empty or near empty), or may slow the delivery rate (when nearly empty or when a pre-set amount of liquid nutritional product has been delivered).

Additional, based on the initial weight of the liquid in the reservoir, the logic 401 may determine the thickness of the liquid nutritional product, and may automatically slow the delivery rate for thicker (heavier) liquid nutritional products and increase the delivery rate for thinner (lighter) liquid nutritional products. This may enable a new mode of feeding in which, after initially delivery most of a meal-time amount of liquid nutritional product, the user may top-off an amount of remaining liquid nutritional product with more liquid nutritional product or even water to thin it out while the pump is operating, thereby allowing for pre-flushing and hydration towards an end of the feeding session.

Another example of the logic 401 functions includes some embodiments which detect occlusions (hose pinched, clog of liquid nutritional product in the tube, jejunum filled, etc.) and/or back pressure on the liquid nutritional product. Such inputs may result in the logic 401 slowing the rate of delivery or even stopping delivery of the liquid nutritional product, to allow the user's body a chance to receive the liquid nutritional product, or to allow the blockage to clear prior to automatically resuming higher-rate delivery of the liquid nutritional product. In this manner, the time for completing a meal-time feeding is minimized by delivering the most liquid nutritional product in the shortest amount of time for which the user's body will accept it.

Further, in embodiments provided with a flow sensor, the logic 401 may be configured to allow the user to select a volume of liquid nutritional product to be delivered, thereby allowing the user to simply fill the reservoir with sufficient liquid nutritional product without measuring it, and then selecting a number of milliliters or ounces to deliver. When the logic 401 determines that amount has been delivered by monitoring the flow sensor and integrating the flow rate over time, the logic 401 may automatically shut off the pump, allowing the user to undock the reservoir, put a lid on it, and store it in a refrigerator for use at the next meal feeding. Features such as this offer a new benefit of reducing the monthly cost of feeding by eliminating wasted or spoiled unused liquid nutritional product.

Conclusion. The terminology used herein is for the purpose of describing particular exemplary embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof, unless specifically stated otherwise.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

1. A high rate liquid food pump system for tube feeding comprising:
a frame;
a motorized peristaltic pump mounted to the frame;
a power supply to the pump;
an on/off control which turns on and off the pump;
a removable reservoir having a top for receiving liquid nutritional product, one or more sides, a bottom with a notch or groove, and a drain in the bottom;
a dock affixed to the frame into which the bottom of the reservoir is received and retained via the notch or groove;
a tube-feeding connector compatible with connecting to an enteral feeding port or flange wherein the tube-feeding connector is selected from the group consisting of a gastrostomy feeding tube (“G-tube” or “button”) connector, a percutaneous endoscopic gastrostomy (PEG) connector, a laparoscopic connector, an open gastrostomy connector, a radiologically inserted gastrostomy (RIG) connector, a nasogastric feeding tube (“NG-tube”) connector, a nasojejunal feeding tube (“NJ-tube”) connector, a gastrojejunal feeding tube (“GJ tube”) connector, and a jejunal feeding tube (“J-tubes”) connector; and
one or more segments of tubing in liquid communication from the drain of the removable reservoir through the pump and to the tube-feeding connector,
wherein the liquid communication between the drain, dock and pump is direct and unimpeded by any other components such that the high rate liquid food pump system is; configured to deliver over one liter of liquid nutritional product per hour from the reservoir to the tube-feeding connector.

2. The high rate pump system of claim 1 further comprising a flow rate control which varies an operating speed of the pump.

3-4. (canceled)

5. The high rate pump system of claim 4 wherein the reservoir is provided with a valve to prevent leakage of the liquid nutritional product while undocked from the system.

6. The high rate pump system of claim 4 further comprising one or more stand structures which prevent the vertically-oriented undocked reservoir from tipping over when rested on a table top or on a shelf.

7. The high rate pump system of claim 4 further comprising a lid attachable to the top of the reservoir.

8. The high rate pump system of claim 1 further comprising control logic configured to receive one or more sensor inputs and to, responsive to the one or more sensor inputs, control at least the on/off control.

9. The high rate pump system of claim 8 wherein the control logic is configured to control the on/off control, or control a flow rate adjustment, or control both the on/off control and a flow rate adjustment, responsive to a level sensor input indicating a level of the liquid nutritional product in a reservoir or carton.

10. The high rate pump system of claim 9 wherein the response to the level sensor input includes automatic flow rate adjustment based on remaining weight of the liquid nutritional product.

11. The high rate pump system of claim 9 wherein the response to the level sensor input includes automatic turning off of the pump responsive to low or no liquid nutritional product remaining in the reservoir or carton.

12. The high rate pump system of claim 8 wherein the control logic is configured to control the on/off control, or control a flow rate adjustment, or control both the on/off control and a flow rate adjustment, responsive to an occlusion detector input.

13. The high rate pump system of claim 8 wherein the control logic is configured to control the on/off control, or control a flow rate adjustment, or control both the on/off control and a flow rate adjustment, responsive to a back pressure sensor input.

14. The high rate pump system of claim 8 wherein the control logic is configured to control the on/off control, or control a flow rate adjustment, or control both the on/off control and a flow rate adjustment, responsive to a timer expiration.

15-20 (canceled)
